(19) United States
(12) Patent Application Publication
(10) Pub. No.: US 2021/0231652 A1
(43) Pub. Date: Jul. 29, 2021

(54) ORGANOMETALLIC LABELS FOR THE DETECTION OF BIOMOLECULES, METHODS OF SYNTHESIS AND PROCESSES FOR CONJUGATING AN ORGANOMETALLIC LABELS TO A BIOMOLECULE

(71) Applicant: George Mason Research Foundation, Inc., Fairfax, VA (US)

(72) Inventors: Lance A. LIOTTA, Bethesda, MD (US); Alessandra Luchini KUNKEL, Burke, VA (US); Amanda N. Haymond STILL, Manassas, VA (US)

(73) Assignee: George Mason Research Foundation, Inc., Fairfax, VA (US)

(21) Appl. No.: 17/059,081

(22) PCT Filed: May 30, 2019
(86) PCT No.: PCT/US19/34629

§ 371 Date: Nov. 25, 2020

Related U.S. Application Data
(60) Provisional application No. 62/678,731, filed on May 31, 2018.
(60) Provisional application No. 62/798,131, filed on Jan. 29, 2019.

(51) Int. Cl. G01N 33/543; G01N 23/2252

(52) U.S. Cl. CPC G01N 33/54346; G01N 23/2252

(57) ABSTRACT

A labeling molecule is provided. The labeling molecule includes a compound of the following formula: CA-(M)-A]n. In the formula, “A” is a label anchor moiety, “M” is an identification moiety, and “CA” is a coordination agent moiety. The label anchor is an organic moiety that binds to a surface of a targeted biomolecule through noncovalent or covalent interactions at one end of the label anchor and binds a coordination moiety. The coordination moiety includes an identification moiety at an end that is distal to the end conjugated to the surface of the targeted biomolecule, and the subscript “n” is 1, 2, 3, 4, or 5.

### CROSS-REFERENCE TO RELATED APPLICATIONS

The present application claims the benefit of priority to U.S. Patent Application No. 62/678,731 filed May 31, 2018 and to U.S. Patent Application No. 62/798,131 filed Jan. 29, 2019, the entire contents of which are incorporated herein by reference.

### STATEMENT OF FEDERAL FUNDING

This invention was made with government support under R33CA206937 awarded by National Institute of Health. The government has certain rights in the invention.

### FIELD

The field of the present disclosure relates to electron microscopy for obtaining high resolution images of cell surfaces, cell components and proteins.

### BACKGROUND

In electron microscopy, especially interesting are images of cellular surfaces of diseased cells and cellular components that play a role in disease pathology, as well as cellular surfaces and components of commercial biological or botanical products. This application provides novel organometallic labels for obtaining high resolution images of cellular surfaces, the detection and quantification of a protein of interest, methods for the synthesis of organometallic labels and processes for selectively conjugating these labels to a select cell, select region of a cellular surface or a cell component or protein of interest. The cells to be imaged are obtainable from tissue biopsy samples, mammalian or bacterial cell cultures, or bulk plant material, for which select subpopulations of cells may be isolated from the sample using Laser Capture Microdissection (“LCM”). The multiple advantages of this technique over other existing methods such as immunohistochemistry (“IHC”) or mass cytometry include high resolution imaging including ultrastructural details, non-ablative techniques such that the sample can be recovered, multiplex analysis for multiple cellular markers, and ease of use.

Laser Capture Microdissection (“LCM”) is an established technology used to isolate tumor cells, or other types of cells, from a heterogeneous piece of tissue under direct microscopic visualization. The isolated cells are used in commercial diagnostic assays, clinical trials, and research studies by pharmaceutical companies and academic institutions. LCM is used by thousands of scientists worldwide. The most popular and useful form of LCM employs a laser beam, or a source of radiation, to heat a thermos-polymer cap that is held against the slice of tissue mounted on a glass slide. FIG. 1A of U.S. Patent Publication No. 2017/0176301 (“the '301 publication”), which is incorporated herein by reference in its entirety for the techniques and apparatuses disclosed therein, illustrates a typical LCM system consisting of a thermoplastic film in contact with the tissue. The plastic film is uniformly impregnated with a dye that absorbs laser energy. The region of the plastic film positioned over the tissue region or cell of interest is selectively heated by the radiation causing this region to melt and embed itself into the tissue segment immediately underneath (FIG. 1B of the '301 publication). When the film is lifted off the tissue the portions of the tissue adherent to the undersurface of the film are ripped free of the tissue section (see e.g., Espina V. et al. (2006) Nature Prot. 1(2):586-603). In addition to the instrument and method described in the 301 publication, other less popular methods and instruments for LCM are available, such as those from Leica or Zeiss (PALM). For the purpose of simplifying discussion, mechanistic details of LCM are described using the most popular instrument design as given in the 301 publication. However, “LCM” should be broadly understood to incorporate all commercially available LCM systems from diverse manufacturers.

### SUMMARY

The multiple advantages of LCM techniques over other existing methods such as immunohistochemistry (“IHC”) or mass cytometry include high resolution imaging including ultrastructural details, non-ablative techniques such that the sample can be recovered, multiplex analysis for multiple cellular markers, and ease of use. A limitation of current LCM methods is that cells of interest must be harvested from the thermoplastic film and then processed to visualize, identify and quantify genes and proteins in the harvested cells. Electron microscopy is most often utilized to obtain high resolution images of cell surfaces, cell components, or proteins. Current methods to detect select features of the cells or proteins, and increase the contrast of the images, requires the biological sample to be stained with heavy metal reagents containing gold, iridium, or osmium. The heavy metals scatter the electrons from the electron microscope, causing the areas of the sample with the heavy metal deposits to show up as bright spots.

However, there are serious disadvantages associated with the use of heavy metals to label proteins and cells. First, heavy metals are very expensive, making this type of imaging very expensive for routine studies. Second, techniques using these heavy metals can be very laborious. Third, the heavy metals are quite toxic, leading to slow and dangerous lab work. Fourth, the detection of cellular components by heavy metals is due to a contrast between the labeled and unlabeled components on the electron micrograph. Since labels of different heavy metals cause similar contrasts, it is hard to tell the difference between different heavy metal labels. Consequently, only one kind of heavy metal probe can be used at any given time with a sample, which severely limits needed multiplex analysis.

The present disclosure provides a novel method of multiplex quantitation and spatial mapping of cellular proteins and nucleic acids within individual tissue cells captured by Laser Microdissection. The largest immediate need for quantitative molecular analysis is in the field of diagnostic pathology and precision medicine where individual cancer or host cells (e.g. immune cells) within a patient's biopsy tissue section and proteins and genes within such cells are detected and quantified. The present disclosure has applications in many different fields including veterinary science, forensic science and plant molecular biology.

The present disclosure relates generally to organometallic labels, methods for the synthesis of such labels and processes for conjugating such labels to a biomolecule. Specifically, the invention relates to metal-containing small organic compounds (labels) that can non-covalently bind to the surface of biomolecules such as proteins.

In at least one embodiment, the disclosure relates to an organometallic label that comprises (a) an identification element; (b) a coordination agent; and (c) a label anchor for binding the label to a surface of a targeted biomolecule. The label anchor according to one embodiment is covalently linked to the surface of a targeted biomolecule. According to another embodiment, the label anchor according to one embodiment is non-covalently linked to the surface of a targeted biomolecule.

According to at least one embodiment, the organometallic label is a water soluble, non-toxic, organometallic compound. In one embodiment, the identification element comprises a transition metal selected from the group comprising titanium, copper, zinc, chromium vanadium, manganese, iron, cobalt, or nickel.

In at least one embodiment, the coordination agent comprises an organic compound having one or more metal chelating groups. Exemplary of such coordinating groups without limitation are polymers of acrylamide, ethylenediaminetetraacetic acid (EDTA), polymers of EDTA, phthalocyanine group, sulfonated phthalocyanine, and the like.

Labels according to at least one aspect of the disclosure enhance visual resolution of the sample and provide a greater throughput of samples analyzed than conventional immunohistochemistry, and mass cytometry. In one embodiment, the coordination of the metal to the metal coordination agent is performed in an aqueous environment, or a solvent comprising a mixture of water or buffer and a suitable water-miscible organic solvent. Coordination of the metal to the coordinating agent comprises contacting a suitable salt of the metal with a solution of the coordinating agent.

In at least one embodiment, the label is covalently attached to a surface residue of a biomolecule. In at least one embodiment, the surface residue has a carboxylic acid group and is coupled to a label having an amino group (—NH₂) or a primary amine group (—NH(R₁)—), to form an amide bond (—C(O)—NH—). When the covalent bond is through a primary amine group, substituent “R₁” is an alkyl, such as a (C1-C4) alkyl group or an aromatic group such as a phenyl, 4-hydroxy phenyl, or 4-amino phenyl group. Alternatively, the label comprises a carboxylic acid group and the surface moiety of a targeted biomolecule comprises an amino group. In this embodiment a reverse amide linkage (—NH—C(O)—) conjugates the label to a surface group on the biomolecule.

The terms “group(s)” and “residue(s)” are used interchangeably throughout this application and refer to a chemical compound, an amino acid, a nucleoside, a nucleotide, or a functional moiety of a chemical compound, amino acid, a nucleoside, or a nucleotide that takes part in chemical conjugation or electrostatic interaction.

In some embodiments the label is covalently linked to a surface group of a biomolecule through a thioether linkage (—S—R₂). In one embodiment, the surface residue has a thiol (—SH) group and is coupled to a label having a iodoacetamide functionalized moiety having the general structure I—CH₂—C(O)—NH—. Thus, the label is covalently linked to a thiol of a surface residue of a biomolecule through a —S—CH₂—C(O)—NH— linkage.

In at least one embodiment, the thioether linkage has the general formula —S-maleimido-CH₂—C(O)—NH—. Such a linkage occurs by contacting a label having an amino group (—NH₂) or a primary amine group (—NH(R₁)—), to a heterobifunctional linker, such as N-α-maleimidoacet-oxysuccinimide ester (AMAS) and contacting the succinimide ester functionalized linker with a sulhydryl group (—SH) of a cysteine.

In at least one embodiment, molecular identification tags (or “barcodes”) having a defined number of the same organometallic labeling molecules conjugated to each other (homo polymer) or a defined number of different organometallic labeling molecules (labels) conjugated to each other (hetero polymer) can be used as molecular identification tags. Using such tags may be advantageous to improve the signal and sensitivity of detection. Alternatively, by using labels comprising different metals, it is possible to tune the wavelength of the signal detected, permitting the simultaneous detection of two or more biomolecules in a single sample.

In at least one embodiment, the organometallic labels are connected to form a polymer that can be used to develop a molecular identification tag specific for a biomolecule based on the ratio of the incorporated identification elements (identification metals).

In at least one embodiment, the organometallic label enhances contrast and enhances the sensitivity of detection by electron microscopy, elemental spectroscopy on a transmission electron microscope, elemental spectroscopy on a scanning electron microscope, or molecular weight on a mass spectrometer. Thus, biomolecules labelled with an organometallic label according to one or more embodiments have higher contrast than unlabeled biomolecules under the same conditions.

According to another embodiment, the organometallic label enhances contrast of the image obtained using scanning electron microscopy.

In at least one embodiment, the identification element of an organometallic label is Ti, V, Cr, Mn, Fe, Co, Ni, Cu, or Zn. The biomolecules labelled with an organometallic label of at least one embodiment are selected from the group consisting of proteins, nucleic acids, carbohydrates, lipids, or any combination of these biomolecules. In one embodiment, the biomolecules are labelled in isolation. According to another embodiment, two or more biomolecules can be labelled simultaneously using different labels or different molecular identification tags.

In at least one embodiment, the biomolecules are incorporated into cell surface structures or interior cellular structures. Labelling of such biomolecules is carried out using the organometallic labels and molecular identification tags of at least one embodiment. The detection of organometallic labelled biomolecules is carried out by electron dispersive spectroscopy that impinges accelerating electrons on to the label causing the organometallic label to emit X-rays of defined wavelengths (Kα, Kβ, or Lα) based on the transition metal (identification moiety) that is coordinated and present in the label.

In at least one embodiment, a method is provided of labeling a target biomolecule comprising (a) a labeling molecule comprising (i) a label anchor moiety; (ii) an identification moiety; and (iii) a coordination agent moiety; and (b) contacting one or more surface residues of a target biomolecule to one or more labeling molecules in a single step, in an aqueous environment.

According to at least one exemplary method, the one or more labeling molecules can comprise the same transition metal (identification moiety) coordinated to the coordination agent moiety or a different transition metal coordinated to the coordination agent moiety.

In at least one embodiment, the labeling molecule is a compound according to Formula I:

[CA-(M)-A]ₙ (Formula I)

For compounds of Formula I “A” is a label anchor moiety, “M” is an identification moiety, “CA” is a coordination moiety, and subscript “n” is 1, 2, 3, 4, or 5.

In yet another embodiment, a method for identifying a single biomolecule or a plurality of biomolecules comprises providing a plurality of different labels, one or more of the labels comprising: i. a label anchor moiety; ii. an identification moiety, wherein the identification moiety of each of the plurality of labels is a different metal; and iii. a coordination moiety. In some embodiments, each of the different labels include the label anchor moiety, identification moiety, and coordination moiety.

According to the o some embodiments, a label anchor can comprise a functional moiety or a functionalized linker for coupling to a surface residue of a biomolecule. Detection of the plurality of labeled biomolecules can be carried out by elemental spectroscopy using a transmission electron microscope, elemental spectroscopy using a scanning electron microscope, or by measuring the molecular weight of the labeled biomolecule using a mass spectrometer.

According to some embodiments, a method for laser capture microdissection includes placing a slide having a transparent and conductive coating so as to be in contact with a sample including a labeled biomolecule, and carrying out electron dispersive spectroscopy on the tissue sample.

### FIGURES

FIG. 1: This figure depicts a schematic workflow that relates to diagnostic pathology according to at least one embodiment.

FIG. 2: This figure illustrates synthesis of metallic nanoparticles using apoferritin protein as a cage.

FIG. 3: This figure illustrates a water-soluble, one-step chemical method for conjugating an organometallic label molecule to an antibody.

FIG. 4: This figure illustrates noncovalent binding of a Fast Blue B naphthionic acid label to thyroglobulin.

FIG. 5A: This figure illustrates amplification of a signal for detection by EDS.

FIG. 5B: This figure illustrates amplification of a signal for detection by EDS.

FIG. 6: This figure shows EDS-detected imaging using molecular identification tags (barcodes).

FIG. 7: This figure illustrates validation of the sensitivity and affinity of organometallic labeling molecule tagged antibodies (MetalloTagged Antibodies).

FIG. 8: This figure provides an overview of Laser Capture Microdissection using caps having a flat surface overlaying the tissue sample of interest.

FIG. 9A: This figure illustrates a cap including a base.

FIG. 9B: This figure illustrates a cap according to at least one embodiment, the cap including feet and a patterned thermoplastic surface configured to contact the tissue of interest.

FIG. 10: This figure illustrates the application of organometallic Labelled Antibody for direct Electron Microscopy Detection of cells captured on the feet of caps, according to at least one embodiment.

FIG. 11: This figure depicts EDS results for an organometallic labeling molecule (MetalloTag) showing a limit of detection to 10 ug/mL on a silicon substratum, according to at least one embodiment.

FIG. 12: This figure depicts LCM cap holders according to at least one exemplary non-limiting embodiment.

FIG. 13: This figure depicts glass slide holders according to at least one exemplary non-limiting embodiment.

### DETAILED DESCRIPTION

The present disclosure relates generally to organometallic labels, methods for the synthesis of such labels and processes for conjugating such labels to a biomolecule. Specifically, the disclosure sets forth embodiments relating to metal-containing small organic compounds (labels) that can noncovalently bind to the surface of biomolecules such as proteins. These small labels are cheap, nontoxic, and water-soluble, so they are easily used in the detection, visualization, and quantitation of cells, cellular surfaces, proteins and genes. The organometallic labels comprise three parts—(a) Identification Element, which is a transition metal selected from the group comprising titanium, copper, zinc, chromium vanadium, manganese, iron, cobalt, or nickel; (b) Coordination Agent—which is an organic compound having one or more metal chelating groups. Exemplary of such coordinating groups without limitation are polymers of acrylamide, ethylenediaminetetraacetic acid (EDTA), phthalocyanine group, sulfonated phthalocyanine, and the like; (c) Label Anchor—an organic moiety that binds to the surface of the targeted biomolecule. The label anchor can bind the biomolecule of interest non-covalently or covalently thereby attaching the label to the surface of the biomolecule through one or more surface residues. The end of the label anchor distal from the biomolecule binds the coordination element. Illustrative label anchors comprise moieties of azoic fast dyes, phthalocyanines, or sulfonated phthalocyanines.

In the context of this disclosure the term “label” refers to unit comprising an identification element, a coordination agent, and a label anchor. In at least one embodiment, the label comprises an identification element, namely a transition metal and a coordination agent, the latter suitably functionalized to directly bind to a surface of the targeted biomolecule. In at least one embodiment, the label binds to a surface of the targeted biomolecule covalently. In at least one other embodiment, the label binds to a surface of the targeted biomolecule noncovalently, for example by one or more electrostatic interactions.

Labels in accordance with at least one embodiment find utility in multiplex quantitation and spatial mapping of cellular proteins and nucleic acids within individual tissue cells captured by Laser Microdissection. Implementations may include identification of diseased versus healthy tissue states, analysis of plant products, or determination of microbial colonization. There is an immediate need in diagnostic pathology and precision medicine to accurately perform a molecular analysis on individual cancer or host cells (e.g., immune cells) within a patient's biopsy tissue section and quantify molecular markers associated with a disease process. Such labels, together with Laser Capture Microdissection (LCM) techniques, may be used to address these deficiencies in the diagnostic pathology and precision medicine markets.

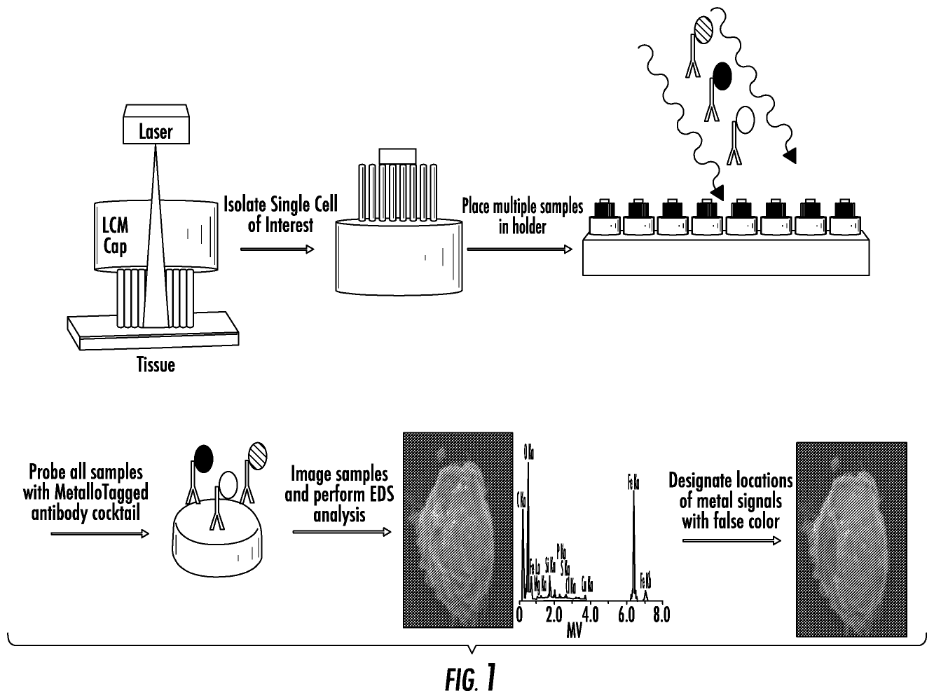

In one embodiment, a pathologist views and remotely marks cells of interest using a computer screen showing the microscopic image of a patient's biopsy (FIG. 1). Individual cells of interest in a tissue biopsy sample can be harvested by LCM. The cap with the harvested cells of interest adhering to its surface is directly loaded into a sample holder that is configured to hold many caps containing adhered cells from different tumor tissue specimens. The entire array of caps containing adherent cells (micro dissection of sample) can be probed as a batch with a cocktail of MetalloTagged antibodies for multiplex analysis. The tagged samples can be imaged by SEM and analyzed by electron dispersive spectroscopy (EDS). Finally, the location of each metal signal can be given a false color designation and mapped onto the SEM image to give detailed information about the location and abundance of a large variety of surface proteins from a single sample. The cells of interest are then remotely captured on to a thermopolymer cap by Laser Capture Microdissection (LCM). Without further manipulation of the captured cells, the individual selected cells of interest are analyzed for a panel of relevant diagnostic analytes. The data from such an analysis is returned to a pathologist, generally within 24 hours and used for diagnosis and personalized therapy.

Analysis is performed as follows. Tissue cells of interest from a pathologic tissue section are captured by Laser Capture Microdissection and displayed on a thermopolymer cap surface. The cap with the selected cells, is incubated with an organometallic label, that is a metal ion dye, alternatively with a plurality of organometallic labels (“barcode” probes), and then inserted into a sample holder of a scanning electron microscope (SEM) equipped with electron dispersive spectroscopy (EDS). EDS detectors such as an Oxford XMaxN SDD or an EDAX Octane Elect SDD have suitable detection limits for this analysis. The micrographic output is a quantitation of each analyte that is overlapped with the captured cell image. Such analysis is rapid, generally requiring less than 1 hour for complete analysis. This technology has higher resolution and throughput than immunohistochemistry (scanning electron microscopy versus light microscopy) and is less expensive than Imaging Mass Cytometry (no need to run mass spectrometry analysis on hundreds of parts of the sample). The ability to polymerize the small organometallic labels allows the manufacture of specific identification tags based on the ratio of different coordinated metals used to manufacture the tag. Such tags can then be used to label different antibodies, proteins, or cell surface structures within a population of cells captured onto the surface of the thermopolymer cap. The use of tags comprising labels of different metals permits assigning regions of “false color” to the image.

An advantage of carrying molecular analysis of analytes using the technique described above is the use of antibodies (or nucleic acid probes) that are conjugated to organometallic labels of transition metal ions. The unique metal containing label permits detection of an analyte by EDS coupled to SEM, rather than fluorimetry which is required for labels containing a fluorophore group. While SEM alone has suggested that surface structures of cancerous cells are altered in ways that can prove diagnostically useful, coupling SEM to detection and localization of proteins of interest within cancer cells is important to enhance our understanding of differential protein expression or aberrant gene regulation in healthy versus aberrant cells.

Indeed, by attaching organometallic labels according to at least one embodiment to selected disease cells attached to the cap of the LCM, a pathologist can locate proteins of interest on cell surfaces without downstream mass spectrometry, the use of toxic heavy metals dyes, and with a resolution and throughput far greater than that achievable with immunohistochemistry.

### Development of the Metallo Label

To detect antibodies used for analyte analysis by EDS, the antibodies are functionalized using an organometallic label of at least one embodiment. The conjugation of an antibody to an organometallic label is rapid and is carried out using water soluble coupling reagents. The organometallic labelled antibodies provide a novel probe for the detection and quantification of analytes in tissue samples of biopsies. By combining different antibodies comprising different organometallic labels and using such a cocktail to label proteins in the same sample, at least one embodiment provides a multiplex analysis of single cells.

In one embodiment, the coordinating agent is a polymer of lysine and N,N-methylene diacrylamide called LYMA. LYMA may be synthesized using the protocol shown in Scheme 1. In some embodiments, LYMA is used in imaging, e.g., as a coordination agent moiety of an organometallic label.

By coordinating a single transition metal, or specific ratios of multiple transition metals, the present inventors can produce large numbers of unique organometallic labels or unique identification tags. As shown in FIG. 2, commercially-available polyacrylamide is one such polymer that can serve as a foundation for subsequent functionalization with EDTA. These EDTA groups can chelate the chosen metal ions, as depicted by in Scheme 1 using copper as an example.

The polymer shown in Scheme 1 below can chelate various transition metal ions, for example copper ions with high efficiencies. Functionalization of a carboxylic acid group of the LYMA polymer permits is conjugation to an amino group of a Lysine reside on the surface of an antibody.

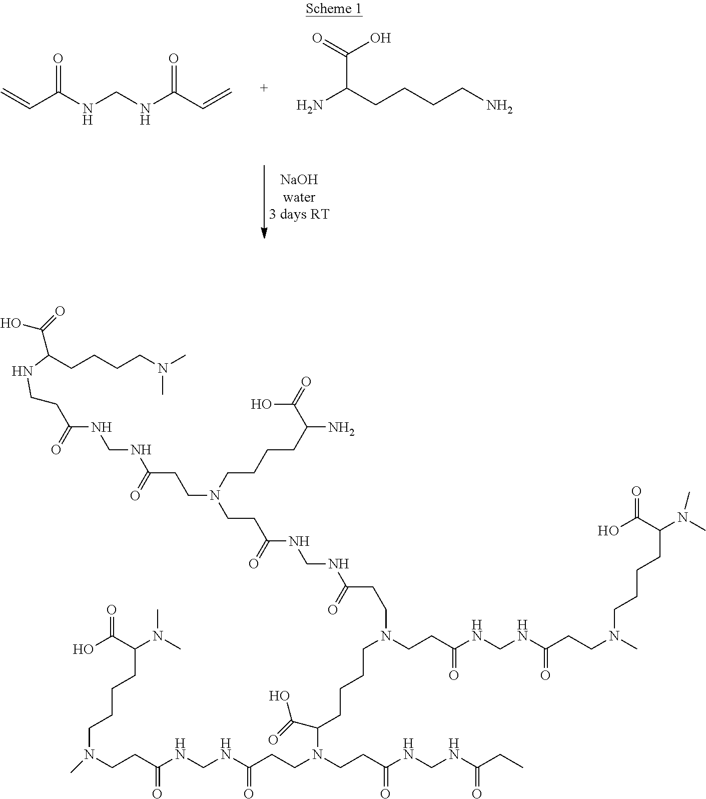 [SMILES: C=CC(=O)NCNC(=O)C=C ; NCCCCC(N)C(=O)O ; CCC(=O)NCNC(=O)CCN(CCC(=O)NCNC(=O)CCN(C)CCCCC(C(=O)O)N(C)C)C(CCCCN(CCC(=O)NCNC(=O)CCN(C)CCCCC(C(=O)O)N(C)C)CCC(=O)NCNC(=O)CCN(CCCCC(N)C(=O)O)CCC(=O)NCNC(=O)CCNC(CCCCN(C)C)C(=O)O)C(=O)O]

Scheme 2 shows another embodiment of a coordinating moiety of a label of at least one embodiment. The highly branched structure of a metal coordinating moiety obtained by polymerization between 1,2-aminoethane (EDA) and EDTA anhydride may have advantages, such as the ability of such a coordinating moiety to incorporate a larger amount of a transition metal and superior purification properties of such polymers.

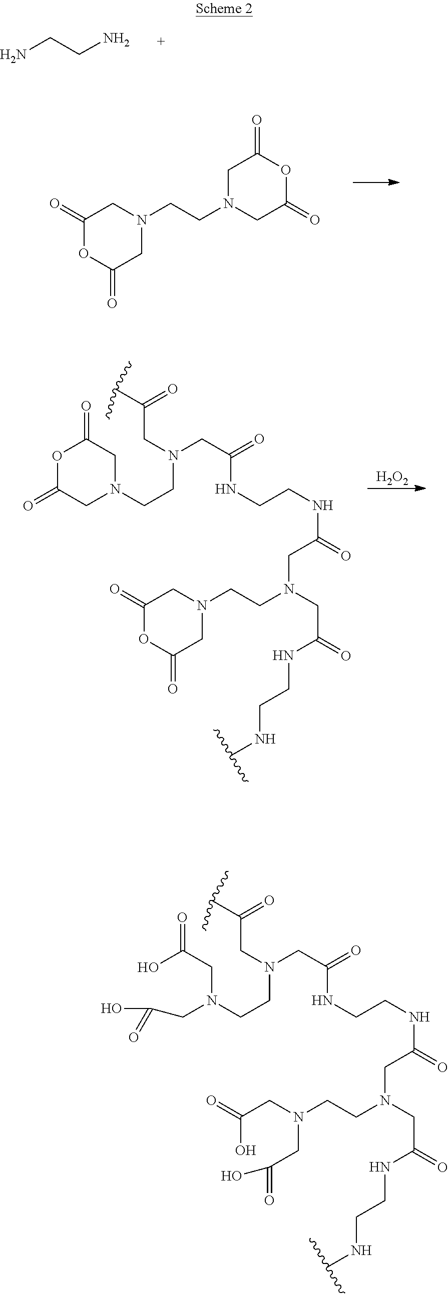

The level of crosslinking in the final polymer is controlled by changing the ratio of 1,2-aminoethane crosslinker to EDTA monomer. In one exemplary polymer synthesis, 4 parts EDA to 1-part EDTA were polymerized to form a highly branched resin-like polymer. Addition of CuCl₂, NiCl₂, or other soluble first-row transition metals salts in high molar excess initiated chelation of the metal ions exclusively through the tertiary amine groups of the repeating polymeric unit. The product polymer incorporates about 5% by weight metal ion which is within the detection limit for low Z (Z<30) elements on commercially available EDS systems.

In yet another embodiment, a metallic nanoparticle is chelated to a coordination moiety so increase the amount of metal incorporation in the label according to at least one embodiment. As stated above, EDTA polymers incorporate metal ions in a range of 5%-7% by weight of polymer. While the limits of detection for commercial EDS systems are about 1% by weight metal, incomplete saturation of the polymer with metals can lead to poor sensitivity of detection. One method to overcome this limitation is to turn to metallic nanoparticles.

Metallic nanoparticles are known in the literature and can be synthesized by loading metal salts into apoferritin, an iron-sequestering protein with a cage-like structure than can hold up to 4500 iron atoms in vivo. Removal of the protein cage by thermal or chemical means following synthesis yields a metallic nanoparticle. FIG. 2 illustrate the use of Apoferritin for the synthesis of transition metal nanoparticles, including Cu, Co, Zn, and Fe. Utilizing similar chemistries, metal nanoparticles for direct labelling of antibodies may be prepared. Such labelled antibodies are distinct from commercially available antibodies labelled with heavy or precious metals for electron microscopy or flow cytometry applications.

Briefly, the synthesis of metallic nanoparticles using apoferritin protein as a cage is carried out by adding a salt of the desired metal ions to a solution of apoferritin. This results in apoferritin uptake of the metal ions and their subsequent oxidation to insoluble crystalline form. Following removal of the outer protein shell by heat denaturation, the metallic nanoparticles can be collected, washed, and functionalized for conjugation to an antibody.

Organometallic Labelling of Antibody

Organometallic labeling molecule according to at least one embodiment must be coupled to an antibody of interest for downstream detection. The method used for coupling a labeling molecule to an antibody may depend on the specific surface residue of the antibody to which coupling is desired the ease, specificity, and efficiency of coupling of a labeling molecule to the antibody. In at least one embodiment, different labeling molecules are coupled to surface residues in discrete, specific regions of the antibody.

According to an embodiment, the free carboxylate groups of an EDA-EDTA polymer or a LYMA polymer is first contacted with N-hydroxysuccinimide (NHS) or sulfo-NHS in the presence of a coupling agent such as 1-ethyl-3-(3-dimethylaminopropyl)carbodiimide (EDC) or 1-cyclohexyl-3-(2-morpholinoethyl)carbodiimide (CMC) to form the corresponding N-hydroxysuccinimidyl ester (NHS) or sulfo-NHS ester as shown in Scheme 3. The resultant NHS-ester activated organometallic labeling moiety is contact with a primary antibody having a surface lysine residue. Conjugation of the organometallic labeling moiety to the antibody is through an amide bond. The use of EDC and CMC coupling groups and NHS or sulfo-NHS reagents permits the entire antibody labeling process to be carried out in an aqueous medium.

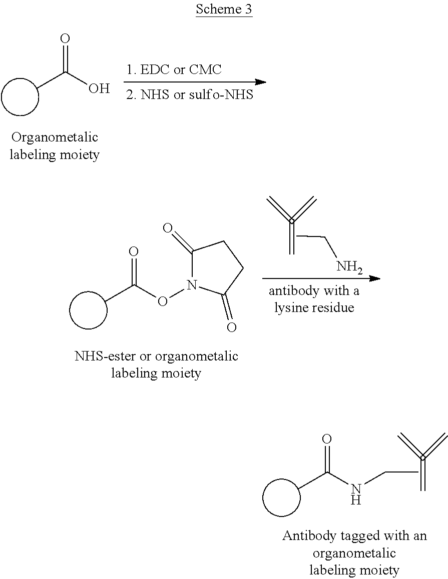

In certain embodiments, if the primary antibody used comprises one or more lysine residues within the antigen binding pocket, tagging an antibody with an organometallic labeling moiety using the NHS-ester chemistries described above may preclude binding of antibody to the antigen and proper antigen recognition. For such antibodies, the organometallic labeling moiety is coupled to a residue in a region of the antibody away from the antigen binding pocket using sulfhydryl coupling chemistry as shown in Scheme 4.

According to this embodiment, one or more free amino groups on the coordination moiety of the organometallic labeling molecule are contacted with the NHS ester of iodoacetic acid to synthesize an iodoacetamide functionalized organometallic labeling moiety. This functionalized organometallic labeling moiety is then contacted with a sulfhydryl group on an antibody to tag the organometallic labeling moiety to the antibody of interest.

In one embodiment, the disulfide bond backbone of the primary antibody of interest is reduced under mild reducing conditions to free sulfhydryl groups, allowing for labelling at a site distinct from the antibody recognition site.

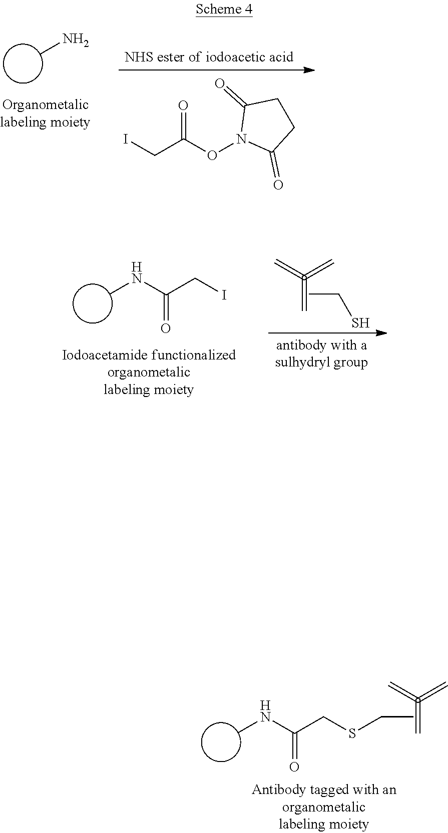

In one embodiment, free metallic nanoparticles are coupled to a primary antibody. Such coupling is carried out by coating the metallic nanoparticles with amphipathic polymers to allow nanoparticle encapsulation by the polymer and to create a coating having free carboxylate groups. These free carboxylate groups can be used to couple the metallic nanoparticle to the primary antibody using the NHS or iodoacetamide coupling chemistry processes described above. This antibody labelling process is illustrated in Schemes 3 and 4.

The coupling of an organometallic labeling molecule of at least one embodiment to a biomolecule such as a protein, a cell surface of a gene can be carried out using Fast Dye Chemistry (azo coupling). FIG. 3 illustrates the coupling of a molecular identification tag comprising different transition metals (triangle, pentagon and circle) connected to each other via a bifunctional moiety called the label anchor (the boxes shown with rounded edges). The label anchor illustrated in FIG. 3 has positively charged azo groups at each end. An example of the bifunctional label anchor is the commercially available azo dye known as Fast Blue B. One of the positively charged azo group of the bifunctional label anchor is coupled to an activated aryl group of a tyrosine residue of an antibody of interest and the other positively charged azo group of the bifunctional label anchor is coupled to the coordination moiety-transition metal complex to provide an organometallic labeling molecule tagged antibody.

The tagging of a biomolecule such as an antibody using the azo coupling chemistries is both novel and simple-to-use. All the reagents are water soluble and coupling of the label to the biomolecule of interest proceeds in one step.

An example of an azo coupling reaction to label an antibody of interest is illustrated in Scheme 5. Using this chemistry, any two groups containing any activated aryl moieties, such as tyrosine residues, can be linked to a compound having two functionally reactive diazonium groups (the diazonium initiator) in a single step in aqueous solution.

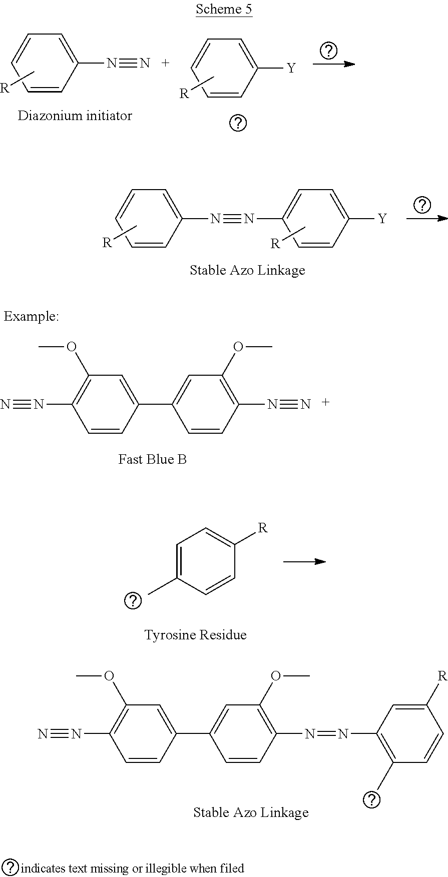

The activated aryl group that couples to the diazonium initiator compound is an aryl ring comprising one or more electron donating group such as —NH₂ or —OH, group. In an exemplary embodiment, Fast Blue B salt, a bifunctional diazonium compound, can be directly coupled to a tyrosine residue on a protein of interest. The second diazonium moiety can be used to covalently link the protein of interest directly to the metallic label, or can couple to an activated aryl moiety such as 4-aminonaphthalene-1-sulfonic acid (naphthionic acid).

In one embodiment the bifunctional diazonium compound is coupled to 4-aminonaphthalene-1-sulfonic acid (naphthionic acid) residues via azo linkages at each end of the Fast Blue B molecule to give a labeling molecule that can bind non-covalently to a protein of interest.

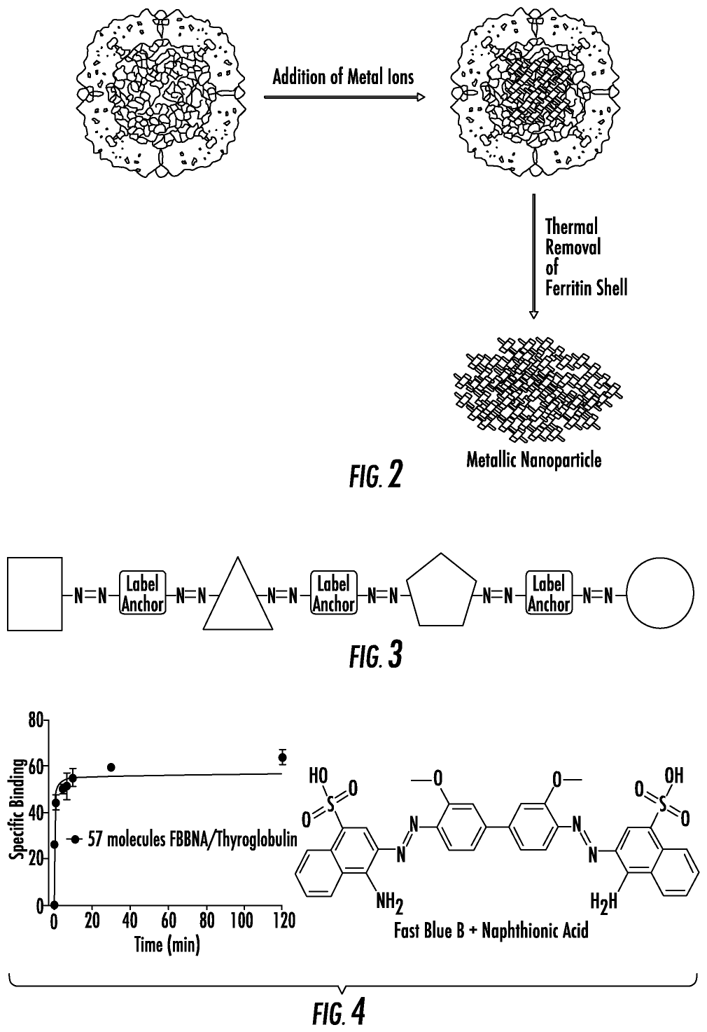

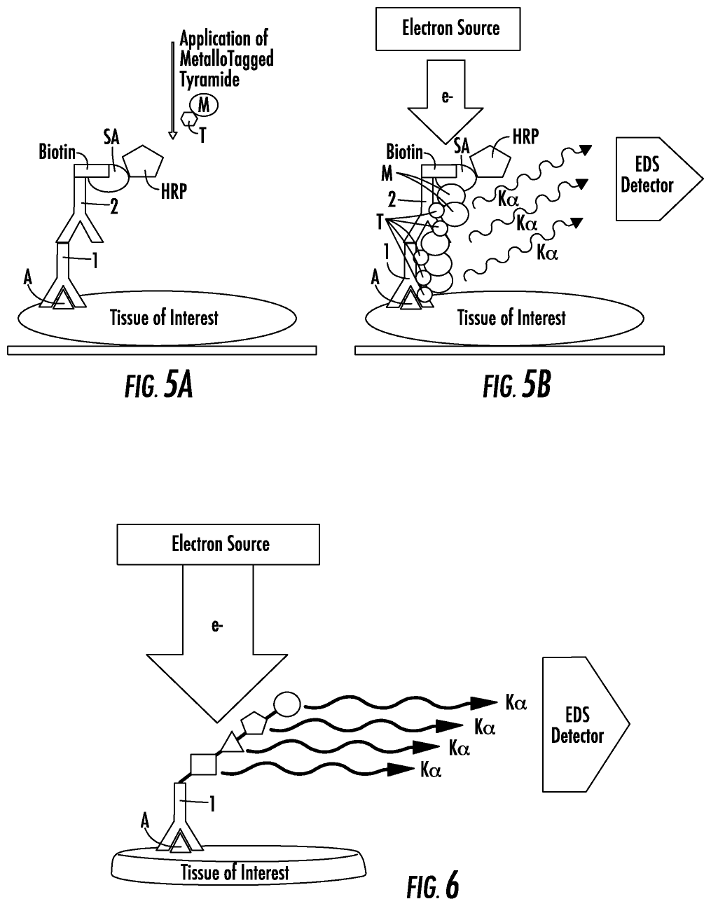

FIG. 4 illustrates the binding curve of a labeling molecule comprising Fast Blue B coupled to 4-aminonaphthalene-1-sulfonic acid (naphthionic acid). As illustrated in FIG. 4, this compound binds noncovalently to the protein thyroglobulin with a specific binding of 57 molecules/molecule of protein. While the label and protein were in contact for a total reaction time of 120 minutes, the curve in FIG. 4 illustrates that equilibrium binding of the label to the protein is rapid, approximately within 5 minutes from the start.

The above visualization method therefore provides researchers with a one-step, water soluble chemical synthesis of a labeling molecule and the use of such a labeling molecule to perform multiplex analysis of analytes in a single cell or a tissue sample with an image resolution comparable to the resolution using electron microscopy. These labeling molecules, the method for labeling a biomolecule as described above, and the high resolution of images address key weaknesses of other available antigen-directed imaging technologies such as MALDI-IMS, CyTOF, or immunohistochemistry (IHC).

### Enzymatic Amplification Chemistry

Signal amplification is necessary to detection and analysis of analytes. Previous work in relation to electron microscopy relied on immunogold catalyzed reporter deposition (CARD) labelling for signal amplification. In this procedure, following labelling of the antigen of interest with a primary antibody, a secondary antibody conjugated to horseradish peroxidase (HRP) is added. Gold nanoparticle-labelled tyramide which is cleaved by HRP into a reactive radical intermediate that immediately deposits on the tyrosine residues in the protein of interest is added following the binding of the secondary antibody conjugated to HRP. This causes the protein of interest to be labelled with gold particles, allowing its visual detection using on the electron micrograph due to density differences from the surrounding material.

However, there are a couple disadvantages to using gold nanoparticles for labeling. First, resolution is limited by the size of the gold particle. If the particle is too small (1 nm or less), it can be difficult to detect from the background electron micrograph image. If the particle is too large, it may obscure details of the labelled biomolecule, and precise localization of the biomolecule within a cell may not be possible. Additionally, only one antigen can be interrogated at a time using a single metal label, namely gold, unless gold particles of very different sizes are used for each amplification reaction. This in turn exacerbates problems encountered with inappropriately sized gold particles.

To overcome these limitations, the present inventors propose the use of electron dispersive spectroscopy (EDS) to determine location of tyramide tagged by an organometallic labeling molecule (“MetalloTags”) of at least one embodiment. In this scheme, multiple amplification reactions can be conducted on the same tissue using different first-row transition metal labeling molecules. Additionally, by using EDS detection rather than visual detection, particles can remain small, and more precise localization can be determined. FIGS. 5A-5B illustrate the deposition of Tyramide tagged with an organometallic labeling molecule and imaging using EDS.

As illustrated in FIG. 5A, the antigen of interest (the triangular symbol labeled A) is detected using a primary antibody (the inverted-Y shape labeled 1 surrounding the triangular symbol labeled A) followed by a biotinylated secondary antibody (the second inverted-Y shape labeled 2). Horseradish peroxidase (indicated by the pentagon-shaped symbol labeled HRP) is conjugated to the antibody complex using streptavidin (the ovoid shape labeled SA). The sample is subsequently treated with tyramide (the hexagon labeled T) tagged using the organometallic labeling molecules according to at least one embodiment (the circle labeled M). The HRP activates the tagged Tyramide to form a reactive radical intermediate that is deposited on the antibody complex and local tissue surface. Following such deposition, the sample is imaged using an electron microscope (e.g., an electron source as shown in FIG. 5B). This causes emission of X-ray radiation with a characteristic wavelength, or Kα line from the organometallic labeling molecules. The X-rays are detectable by an EDS detector to identify the metal(s) present.

If signal amplification is not needed, EDS detection can be used directly. For added sensitivity, molecular identification tags (“barcode”) comprising multiple different organometallic labeling molecules according to at least one embodiment may be used to tag a single antibody, as shown in FIG. 6. As illustrated in this figure, a biomolecule of interest, such as an antibody (the inverted Y labeled 1), is labelled with a small polymeric metal label, a molecular identification tag. The labeled antibody is applied to the surface of a tissue to detect a biomolecule of interest (the triangle labeled A). The tissue containing labeled antibody is washed to reduce nonspecific binding. The tissue sample is then placed in an EDS-equipped electron microscope and imaged. The EDS detector picks up the characteristic Kα, Kβ, or Lα X-rays emitted by the metallic label and allows for localization of the biomolecule of interest. As mentioned above, multiple metallic labelling molecules can be polymerized to generate a unique molecular identification tags (barcodes).

The sensitivity of detection using the organometallic labeling molecules (MetalloTag) or molecular identification tags in accordance with at least one embodiment can be performed using a modified dot-blot to quantify the amount of protein and antibody required for detection of a reproducible quantifiable signal. To start, the organometallic labeling molecule tagged antibody is contacted with an antigen the antibody-antigen conjugate is examined using traditional Western blotting of cell lysates. This ensures that labeling of the primary antibody with the MetalloTag does not significantly affect the affinity or specificity for the antibody to the antigen of interest, when compared to the unconjugated primary antibody.

Once the labeled antibody's affinity and specificity are confirmed, the MetalloTagged antibodies is spotted directly onto a solid support for initial detection using EDS. Samples can be imaged by SEM under low vacuum mode (such as that available on a Tescan MAIA3 instrument, JEOL JSM-IT500, JEOL JCM-6000Plus NeoScope made by JEOL USA of Peabody, Mass., or a similar system having a low vacuum mode) to prevent charging on the uncoated samples. Additional conductive mounting strategies may be pursued to ensure reduction in charging, including imaging samples directly on glass microscope slides coated with a transparent, conductive coating. Standard curves indicating the linear range of concentrations of antibody needed for appropriate detection may be generated and used to determine required final antibody concentrations.

Using this standard curve, antigen may be spotted directly on to solid supports (e.g., nitrocellulose films or membranes), and probed with a MetalloTagged antibody, and the film can be washed to remove excess MetalloTagged antibody. The washed film is then placed onto an electron microscope sample holder and the antibody detected using EDS. Accordingly, the minimum protein concentration may be determined for detection using the MetalloTagged antibody under conditions that can cause loss of protein, MetalloTagged antibody during the washing of the solid support.

FIG. 7 illustrates an exemplary process to validate the sensitivity and affinity of the MetalloTagged antibody. The process is completed iteratively for each optimization of the MetalloTag chemistry until sufficient signal is generated with limited nonspecific binding under reasonable concentrations of antibody and antigen. Panel (A) in FIG. 7 shows a Western blot analysis to confirm if the MetalloTagged antibodies bind with specificity to the antigen of interest. Panel (B) of FIG. 7 is a dot-blot analysis to examine the metal composition of the MetalloTag via EDS, following confirmation by Western Blot that the MetalloTagged antibody retains binding activity similar to the untagged antibody. Standard curves may be used to determine a desired concentration of antibody for appropriate signal.

Panel (C) of FIG. 7, a dot-blot prepared and analyzed using EDS to determine the concentration of antigen required for appropriate signal. Prior to the dot-blot analysis Western Blot is used to confirm that the MetalloTagged antibody has specificity for the antigen and sufficient metal incorporation for signal detection. The MetalloTagged antibody is validated after performing the tests described in Panels (A)-(C) and can be used for detection of analytes in cell samples.

### Additional Implementations of LCM Techniques

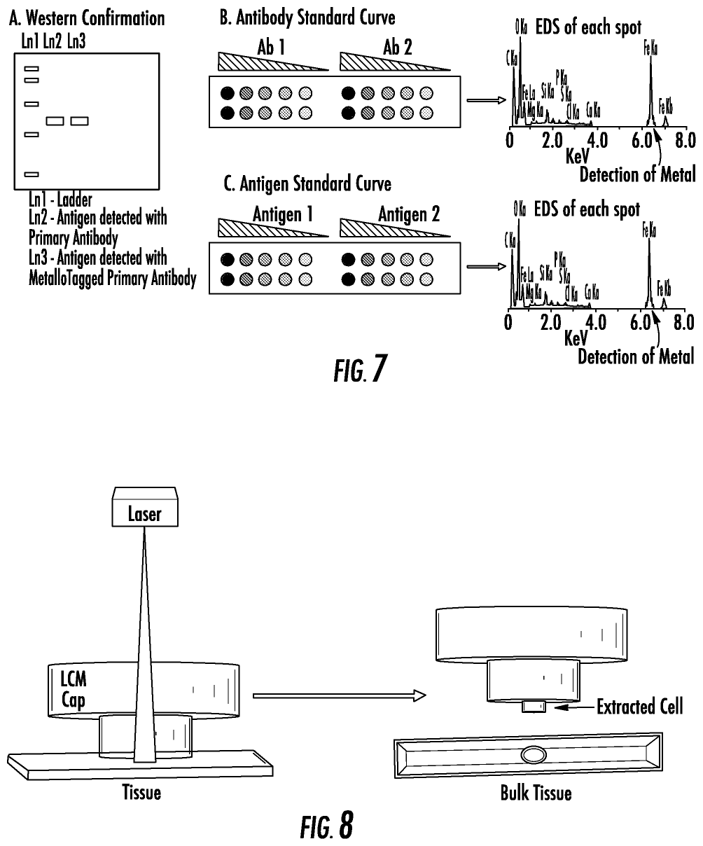

Imaging of cells and cellular components as well as the detection and quantitation of analytes in biological tissue or cells using the organometallic labeling molecules (MetalloTags) of certain embodiments depends on techniques to isolate single or subpopulation of cells of interest. Laser capture microdissection (LCM) provides a precise method for isolating individual cells or a subpopulation of cells, for example, cancer cells from a tissue sample having a heterogeneous population of cells. Single cells are selected by a pathologist viewing an image of the tissue biopsy sample on a screen and isolating the selected cells using a laser that is focused onto the tissue sample through a thermolabile polymer cap placed over the tissue sample. When exposed to the laser light, the cap melts onto the surface of the cell of interest immediately below forming a film to which the selected cells adhere. This permits the selected cells, now adhered to the cap, to be plucked from the bulk sample as the cap is removed. Some LCM caps have a flat surface that overlays the tissue sample, which is fixed on a silicon dioxide glass microscope slide. FIG. 8 illustrates the design of certain caps and microdissection protocol for isolating select cells.

Despite the power and utility of LCM, the method can be further improved. Currently available LCM methods show some variability in the capture efficiency of cells from the desired region of the tissue caused by a reduced adhesive force on the top of the tissue section, that is between the tissue and the film and an increased adhesive force between the tissue section and the glass slide substratum. The resolution of LCM systems can be improved, for example, by addressing both forces. Namely, resolution may be improved by increasing the affinity of the cap for the tissue, and decreasing the affinity of the glass slide substratum for the tissue. Because the presently available caps have a flat surface overlaying the tissue sample, the film melts against the tissue in a variable manner depending on the focus of the laser beam, the surface contours, and the wavelength of the light. Thus, an area spanning many tissue cell diameters is often captured, limiting the resolution and preventing the precise capture of single cells or components of cells.

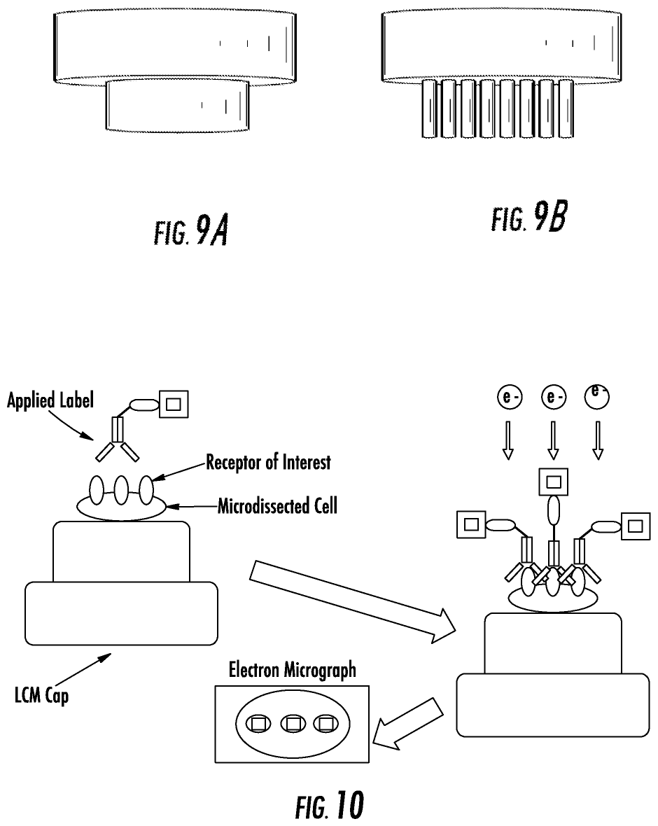

The cap geometry shown in FIG. 9A includes a solid base. In contrast, the cap geometry shown in FIG. 9B includes “feet” rather than a solid “base.” The inclusion of feet to produce a patterned surface is believed to improve cell capture efficiency with a high degree of fidelity of capture for cells of interest. Incorporating “feet” into the cap design accounts for the concern that tissue surfaces are not perfectly flat but contain microscopic dips and grooves. A flat cap therefore cannot contact tissue surfaces in the grooves and dips, that is, at multiple depths. A cap design with “feet” however, increases the contact surface area and allows for movement of the individual feet into the grooves and dips where it can contact tissue surfaces and the cells contained therein.

In at least one embodiment, the cap design, including the size of the “feet”, the density of the “feet”, and the geometry of the feet (cylindrical, rectangular, etc.) was analyzed for effects on capture efficiency without sample distortion. Cap manufacture starts by preparing a metallic mold using focused ion beam (FIB) ablation of a metal sample. Commercially available FIB-SEMs have CAD software and automation to allow for precise nanomachining of the cap mold from the bulk metal, and allow for detailed geometry, density, and size of the “feet” to be considered. Each mold may be used to manufacture many LCM caps using thermolabile polymer.

In one embodiment of the cap with feet, the thermolabile polymer may be impregnated with different dyes that absorb IR radiation. By combining dyes of slightly different lambda max absorbance values within the cap, absorption of the incoming IR radiation may be controlled, allowing for control over the selective swelling and melting of the cap “feet” as they make contact and become stuck to the tissue surface and/or cells of interest.

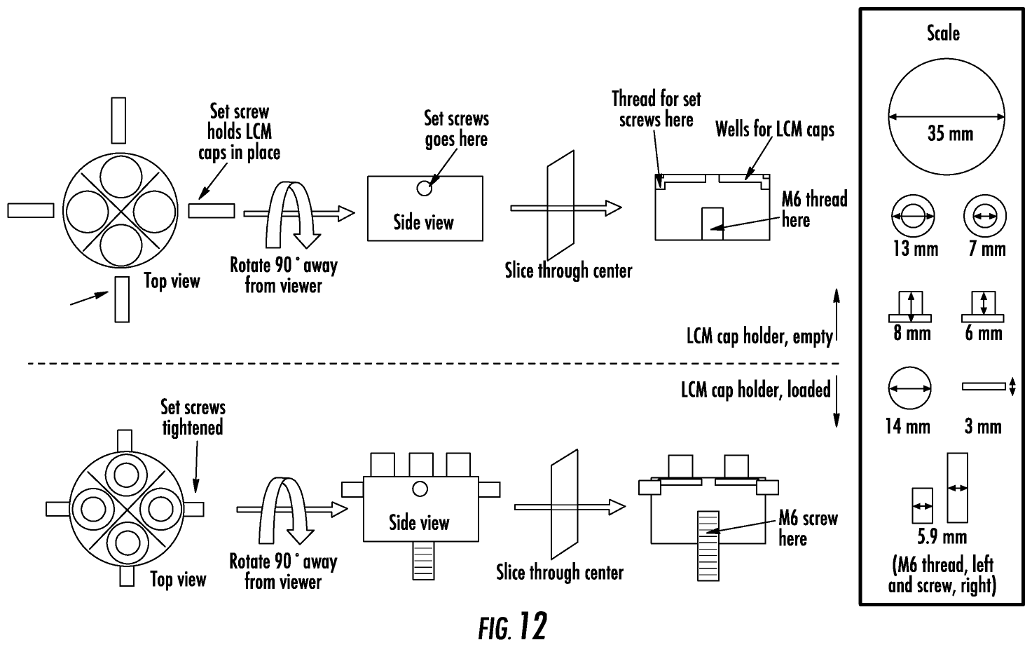

One or more caps according to the aforementioned embodiments may be held in a LCM cap holder structured to accommodate the one or more caps. FIG. 12 depicts LCM cap holders in accordance with at least one embodiment. In some embodiments, an LCM cap holder is structured to be compatible with a stage adapter so as to be utilized in connection with a desktop SEM system, such as the JEOL Neoscope described above. In particular, an adapter such as the EM-Tec JV40 stage adapter kit made by Rave Scientific of Somerset, N.J. may be used in some embodiments. The stage adapter kit comprises a disc insert with a fastener (e.g., a size M4 female thread screw), a stage adapter pillar that is adjustable in height with a fastener or fastener assembly (e.g., a male M6 screw thread topped by an M4 screw), and a locking ring (e.g., an M6 ring that mates with the M6 screw). In some embodiments, an LCM cap holder may include one or more wells dimensioned to secure an LCM cap therein.

As shown in FIG. 12, in at least one embodiment, an LCM cap holder is a substantially disc-shaped holder comprising a plurality of individual wells, and each well may be dimensioned to accommodate a respective cap. For example, in some embodiments, the LCM cap holder may comprise four wells located at equal intervals from each other. A set screw may be provided with each well to keep the LCM cap in place. An M6 thread may be used to secure the LCM cap holder to a stage. When the cap is placed in the holder, the projections or feet of the cap, as shown in FIG. 9B, project upward from the well so as to extend beyond an upper surface of the holder, as seen in FIG. 12. Upon placement of the samples in the LCM cap holder or glass slide holder, the detection of organometallic labelled biomolecules may then be carried out by electron dispersive spectroscopy (EDS).

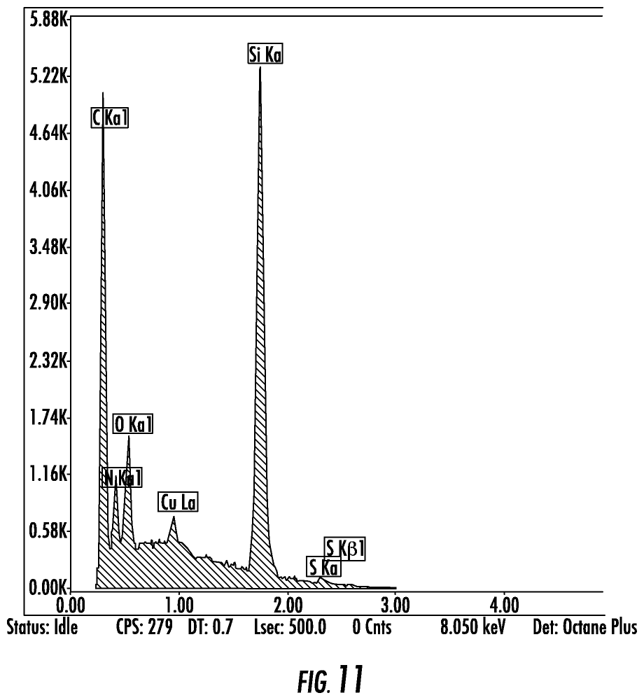

By incorporating the above-described cap designs with EDS and the imaging techniques described above, a SEM image of a cell may be produced, which is captured more efficiently with an LCM cap according to at least one embodiment, imaged using an EDS-technique developed for SEM to provide a micrograph of the cell surface or protein of interest using the organometallic labeling molecules (MetalloTags) of certain embodiments, as shown in FIG. 10.

In FIG. 10, the organometallic labelled antibody to the receptor of interest (orange) is applied and studied under electron microscopy. Using electron dispersive spectroscopy (EDS), the cells of interest can be examined for distinct metallic signatures indicating the location of the antibody. An electron micrograph showing the spatial locations of the receptors of interest—labelled using the MetalloTags comprising first row transition metal (metals represented by yellow squares indicating locations of receptors in electron micrograph depiction)—is generated.

In at least one embodiment, a patterned thermoplastic film comprises (i) a first surface; and (ii) a plurality of projections (“feet”) attached to and extending outwardly from the first surface, wherein the projections form a pattern on the thermoplastic film. In at least one embodiment, the projections are formed on the thermoplastic film using a photolithography mold that is applied to the surface of a thermopolymer mounted on a cap.

In at least one embodiment, the projections are micropillars, micro projections, hydrogel microspheres, and/or microneedles that are attached to, continuous with, or integrally formed with the thermoplastic film layer. In a further aspect, the projections form an array on a surface of the micropatterned thermoplastic film. In some aspects, the projections are formed on a single surface of a planar or substantially planar thermoplastic film layer, and the opposing surface of the substantially planar thermoplastic film layer can be substantially smooth or lacking projections. The projections can extend outwardly from the first surface of the patterned thermoplastic film layer that is perpendicular or substantially perpendicular to the first surface.

In at least one aspect, the micropatterned thermoplastic film can comprise an array of projections that cooperate to define a pattern on a surface of the film. In this aspect, the projections of the array can comprise micropillars, micro projections, hydrogel microspheres, microneedles, or combinations thereof.

As used herein, the term “micropillar” refers to a projection that, prior to activation, has a consistent or substantially consistent outer diameter along its length.

As used herein, the term “microsphere” refers to a projection that, prior to activation, has a generally rounded appearance, including spherical, substantially spherical, ovoid, and substantially ovoid shapes.

As used herein, the term “microneedle” refers to a projection that, prior to activation, has a diameter that decreases (optionally, consistently decreases) moving away from the first surface of the patterned thermoplastic film. According to some embodiments, the “micropillars” and “microneedles” can have any desired cross-sectional shape, including, for example and without limitation, circular, square, rectangular, triangular, oval, elliptical, trapezoidal, pentagonal, hexagonal, heptagonal, or octagonal shapes. Optionally, a microneedle can have a pointed distal tip.

In at least one aspect, each projection of the plurality of projections can be a micropillar. In another aspect, each projection of the plurality of projections can be a microsphere. In yet another aspect, each projection of the plurality of projections can be a microneedle.

In at least another aspect, the plurality of projections can comprise at least one micropillar and at least one microneedle. In on embodiment, the plurality of projections can comprise at least one micropillar and at least one microsphere. In still another aspect, the plurality of projections can comprise at least one microsphere and at least one microneedle. In certain embodiments, the plurality of projections can comprise at least one micropillar, at least one microneedle, and at least one microsphere.

In at least one embodiment, the projections of the micropatterned thermoplastic film can have a width or outer diameter ranging from about 1 nm to about 10 mm and a length ranging from about 1 nm to about 10 mm. The length of each projection can correspond to the height of the projection relative to the surface of the micropatterned thermoplastic film. Exemplary width or outer diameter and the length of each projection can both range from about 100 nm to about 1 mm, about 100 nm, about 200 nm, about 300 nm, about 400 nm, about 500 nm, about 600 nm, about 700 nm, about 800 nm, about 900 nm, about 1 μm, about 10 μm, about 20 μm, about 30 μm, about 40 μm, about 50 μm, about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm, about 190 μm, about 200 μm, about 300 μm, about 400 μm, about 500 μm, about 600 μm, about 700 μm, about 800 μm, about 900 μm, or about 1 mm, or can fall within a range defined between any two of these listed values. Optionally, in exemplary aspects, the length of each projection 72 can be greater than the width or outer diameter of the projection. In one exemplary aspect, the projections 72 can each have a diameter of about 100 μm, and the projections can be fabricated such that each of the projections has a consistent (equal or substantially equal) height ranging from about 10 μm to about 100 μm.

During fabrication, the projections can be fabricated to have any desired spacing relative to other projections (e.g., adjacent projections) within the array. In some aspects, the projections are equal or substantially equally spaced within the array (measured between center points of the respective projections). For example, the projections can be organized in columns, rows, or combinations thereof, in which each projection is spaced from adjacent projections by a consistent distance, such as, for example and without limitation, a distance ranging from about 10 μm to 1 mm and, more preferably, from about 10 μm to about 200 μm, or about 100 μm. However, in other aspects, the projections can be fabricated to have a variable spacing, with at least one projection being closer to some adjacent projections than others. In these aspects, portions of the array can have a higher concentration of some projections than other projections.

In at least one embodiment, micropatterned thermoplastic film, such as an EVA film, used in LCM techniques comprises an absorptive substance. The absorptive substance can include an absorptive dye. This dye can be either a broad band absorptive dye or a frequency specific absorptive dye. For example, the absorptive dyes can include one or more of: tin(IV) 2,3-naphthalocyanine dichloride; silicon(IV) 2,3-naphthalocyanine dihydroxide; silicon (IV) 2,3-naphthalocyanine dioctyloxide; and vanadyl 2,11,20,29-tetra-tert-butyl-2,3-naphthalocyanine. The absorptive dye can be an infrared napthalocyanine dye, available from Aldrich Chemical Company. Also, the absorptive substance can include a plurality of fullerenes (i.e., Bucky Balls, e.g., C60). An absorptive substance can have a strong absorption in the 800 nm region, a wavelength region that overlaps with laser emitters used to selectively melt the film. The absorptive substance is mixed with the melted bulk plastic at an elevated temperature. The thermoplastic comprising the absorptive substance is then manufactured into a film using standard film manufacturing techniques. The dye concentration in the plastic can be about 0.001 M.

Further, in some embodiments, the surface of the glass slide substratum may be treated by coating the surface with a transparent or semitransparent conductive substrate that melts or softens under subjection to the IR laser beam of the LCM machine (i.e., when irradiated with electromagnetic energy or wavelengths in the UV or IR spectrum). The transparent, conductive coating can be a metallic, polymeric, or organic coating with the property that it is transparent and can be melted or softened by subjection to IR laser light of the appropriate wavelength. This allows the glass slide substratum to easily dissociate from the tissue section of interest, improving capture efficiency of LCM. By incorporating an additional coating on the glass slides, such embodiments effectively ameliorate the concern that the affinity of the tissue section of interest for the glass slide is too great to be completely overcome by the affinity of the LCM cap to the tissue section, which may otherwise contribute to tearing or incomplete capture of the cells of interest.

The slides which are used in LCM according to some embodiments were treated with different coating materials to evaluate their effects on capture efficiency without sample distortion. Coated slides were compared for capture efficiency versus cells micro-dissected from uncoated silicon dioxide microscope slides.

Some photovoltaics rely on coated glass slides as substrates for the fabrication of polymeric semiconducting solar cells. Glass slides with transparent, conductive coatings are commercially available, as are slides with semitransparent conductive coatings. The coatings function as n-type semiconducting materials for a variety of optical and electro-optical applications.

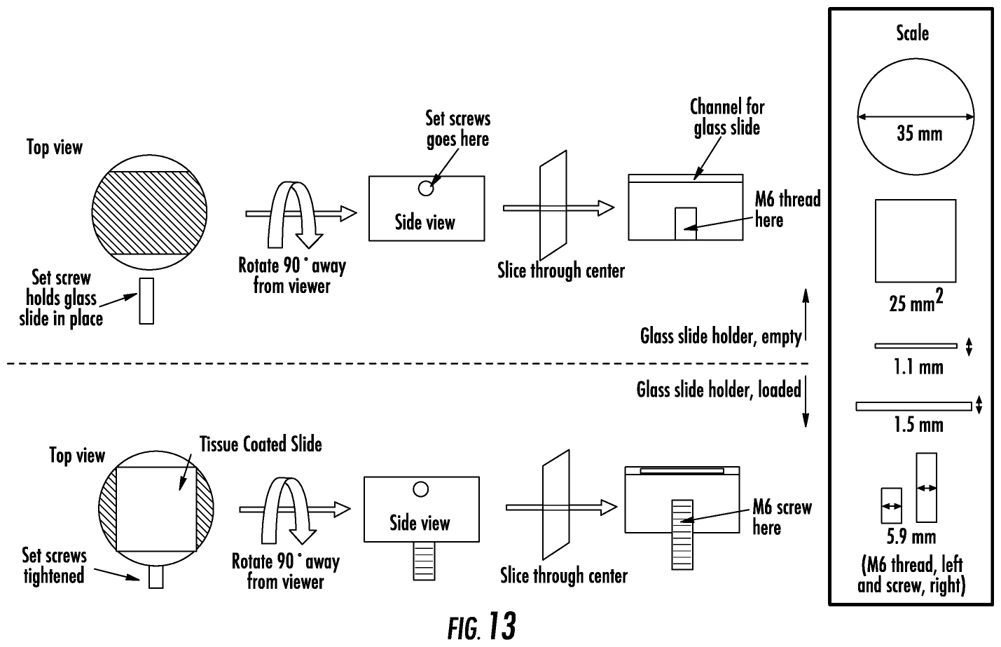

FIG. 13 depicts LCM glass slide holders according to one or more embodiments. The glass slide holder according to at least one embodiment includes a shallow channel disposed on a substantially disc-shaped base. The channel is dimensioned so as to accommodate the glass slide therein so as to securely retain the glass slide in place. The channel is positioned at the top of the base. At the bottom of the base, a fastener (e.g., the M6 thread) may be positioned to attach the glass slide holder to the stage.

In at least one embodiment, the transparent, conductive coating may be a transparent metal oxide such as indium tin oxide (ITO), indium cadmium oxide, zinc oxide (AZO), or indium zinc oxide (IZO). In some embodiments, the coating may be provided on one or both sides of the slide.

In at least one embodiment, the transparent, conductive coating may be a conductive polymer such as poly(3,4-ethylenedioxythiophene) [PEDOT] or poly(3,4-ethylenedioxythiophene) polystyrene sulfonate [PEDOT/PSS].

In at least one embodiment, the transparent conductive coating is a coating with an impregnator, e.g., a coating impregnated with carbon nanotubes to provide conductivity. Impregnated coatings may include previously listed transparent conductive materials (e.g., ITO, AZO or IZO) or another transparent resin, polymer, or silicon-based adhesive.

Slides with conductive coatings are utilizable in some embodiments not only to improve capture efficiency of LCM, but to reduce charging of samples imaged by electron microscopy. In at least one embodiment, microdissected samples are imaged directly on the LCM cap. In one or more other embodiments, microdissected samples are imaged on glass slides with conductive coatings to reduce charging effects.

In some embodiments, the substratum material may be determined without detection of a metallotag. FIG. 11 depicts an EDS spectral image for a MetalloTag imaged directly on a silicon chip substratum. The limit of detection was 10 ug/mL MetalloTag, consisting of 7 wt % copper. Substratum material was detected via EDS, but did not overlap with the detection of the MetalloTag.

In at least one embodiment, biological samples of interest are micro-dissected using LCM from bulk samples on a slide coated with transparent, conductive material as described above and imaged directly on LCM cap after treatment with organometallic labels.

In at least one embodiment, biological samples are imaged directly on slides coated with transparent conductive coatings as described above, after treatment with organometallic labels.

Further, in some embodiments, biological samples of interest are micro-dissected using LCM from bulk samples on a silicon dioxide microscope slide and imaged directly on LCM cap following treatment with organometallic labels.

Unless otherwise defined, all technical and scientific terms used herein have the same meaning as commonly understood by one of ordinary skill in the art to which this invention belongs.

The invention illustratively described herein may suitably be practiced in the absence of any element or elements, limitation or limitations, not specifically disclosed herein. Thus, for example, the terms “comprising,” “including,” “containing,” etc., shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

All publications, patent applications, patents, and other references mentioned herein are expressly incorporated by reference in their entirety, including all formulas and figures, to the same extent as if each were incorporated by reference individually. In case of conflict, the present specification, including definitions, will control.

What is claimed is:

1. A labeling molecule comprising:
a compound of Formula I:
[CA-(M)-A]ₙ
wherein
i. “A” is a label anchor moiety;
ii. “M” is an identification moiety; and
iii. “CA” is a coordination agent moiety;
wherein
the label anchor is an organic moiety that binds to a surface of a targeted biomolecule through noncovalent or covalent interactions at one end of the label anchor and binds a coordination moiety comprising an identification moiety at an end that is distal to the end conjugated to the surface of the targeted biomolecule, and
wherein subscript “n” is 1, 2, 3, 4, or 5.

2. The labeling molecule according to claim 1, wherein the label anchor is non-covalently bound to a surface group of a biomolecule or covalently bound to a surface group of a biomolecule.

3. The labeling molecule according to claim 2, wherein the biomolecule is selected from the group consisting of proteins, nucleic acids, carbohydrates, lipids, and combinations thereof.

4. The labeling molecule according to claim 1, wherein the label anchor moiety couples to the coordination agent moiety via an azo link.

5. A composition of claim 1 where the labeling molecule comprises a cleavable moiety.

6. The labeling molecule according to claim 1, wherein labeling molecule permits the detection of the target biomolecule by elemental spectroscopy using a transmission electron microscope, elemental spectroscopy using a scanning electron microscope, or by molecular weight analysis using a mass spectrometer.

7. The labeling molecule according to claim 1, wherein the identification moiety is a transition metal, selected from the group consisting of Ti, V, Cr, Mn, Fe, Co, Ni, Cu, and Zn.

8. The labeling molecule according to claim 1, wherein the coordination agent moiety contains metal chelating groups to bind the identification moiety.

9. The labeling molecule according to claim 9, wherein the coordination agent moiety is selected from the group consisting of ethylenediaminetetraacetic acid group, an ethylenediaminetetraacetic acid resin, a sulfonated phthalocyanine ring, or a polyethyleneimine resin.

10. The labeling molecule according to claim 1, wherein subscript “n” in the compound of Formula I is 2, 3, 4, or 5.

11. The labeling molecule according to claim 10, wherein subscript “n” is 2 or 3 and the labelling molecule is a polymer comprising more than one coordination agent moiety, and identification elements.

12. The labeling molecule according to claim 10, wherein the polymer is a molecular identification tag that emits a Kα, Kβ, or Lα X-ray radiation that is specific to the ratio of incorporated identification elements.

13. A labeling molecule comprising:
i. an identification moiety; and
ii. a coordination agent moiety;
wherein
the coordination agent moiety comprises free amino (—NH₂) and free carboxylate (—COO⁻) groups which bind to a residue on a surface of a targeted biomolecule through noncovalent or covalent interactions.

14. The labeling molecule according to claim 13, wherein the coordination agent moiety is selected from the group consisting of ethylenediaminetetraacetic acid group, an ethylenediaminetetraacetic acid resin, a sulfonated phthalocyanine ring, or a polyethyleneimine resin.

15. A method of labeling a target biomolecule comprising:
providing a labeling molecule comprising (i) a label anchor moiety; (ii) an identification moiety; and (iii) a coordination agent moiety; and
contacting one or more surface residues of a target biomolecule to one or more labeling molecules in a single step, in an aqueous environment,
wherein the one or more labeling molecules comprise the same transition metal coordinated to the coordination agent moiety or a different transition metal coordinated to the coordination agent moiety.

16. The method of claim 15, wherein a plurality of target biomolecules is labeled.

17. The method of claim 16, wherein two or more of a plurality of target biomolecules are labeled with a different labeling molecule.

18. The method of claim 15, wherein the anchor moiety of the labeling molecule is covalently or non-covalently linked to the target biomolecule

19. The method of claim 15, wherein the labeling molecule is a compound according to Formula I:
[CA-(M)-A]ₙ (Formula I)
wherein
i. “A” is a label anchor moiety;
ii. “M” is an identification moiety; and
iii. “CA” is a coordination moiety; and
subscript “n” is 1, 2, 3, 4, or 5.

20. A method for identifying a single biomolecule or a plurality of biomolecules comprising:
a. providing a plurality of different labels comprising one or more of:
i. a label anchor moiety;
ii. an identification moiety, wherein the identification moiety of each of the plurality of labels is a different metal; or
iii. a coordination moiety;
wherein the label anchor comprises a functional moiety or a functionalized linker for coupling to a surface residue of a biomolecule; and
b. detecting the plurality of labeled biomolecules by elemental spectroscopy using a transmission electron microscope, elemental spectroscopy using a scanning electron microscope, or by measuring the molecular weight of the labeled biomolecule using a mass spectrometer.

21. The method according to claim 20, wherein labelling increases contrast of at least one labeled biomolecule compared to an unlabeled molecule in a scanning or transmission micrograph.

22. A method for laser capture microdissection, comprising:
placing a slide having a transparent and conductive coating so as to be in contact with a sample including a labeled biomolecule, and
carrying out electron dispersive spectroscopy on the tissue sample.

23. The method of claim 22, wherein the coating is one selected from the group consisting of a metallic coating, a polymeric coating, or an organic coating.

24. The method of claim 22, wherein the coating comprises a transparent metal oxide.

25. The method of claim 24, wherein the transparent metal oxide is indium tin oxide, indium cadmium oxide, zinc oxide, or indium zinc oxide.

26. The method of claim 22, wherein the coating comprises a conductive polymer.

27. The method of claim 26, wherein the conductive polymer is poly(3,4-ethylenedioxythiophene) or poly(3,4-ethylenedioxythiophene) polystyrene sulfonate.

28. The method of claim 22, wherein the coating further comprises an impregnator.

29. The method of claim 28, wherein the impregnator includes carbon nanotubes.

30. The method of claim 22, wherein the coating comprises a n-type semiconductor.
